(19) United States
(12) Patent Application Publication
(10) Pub. No.: US 2023/0149635 A1
(43) Pub. Date: May 18, 2023

(54) Applicator for Implant Insertion

(71) Applicant: Lupin Inc., Baltimore, MD (US)

(72) Inventors: Richard J. Holl, Baltimore, MD (US); Tomas Matusaitis, Chicago, IL (US)

(21) Appl. No.: 17/983,478

(22) Filed: Nov. 9, 2022

Related U.S. Application Data
(60) Provisional application No. 63/278,528, filed on Nov. 12, 2021.

(51) Int. Cl. A61M 5/32; A61M 5/46

(52) U.S. Cl. CPC A61M 5/3293; A61M 5/46; A61M 5/3202

(57) ABSTRACT

The present invention provides an applicator for insertion of drug delivery device for subdermal administration of contraceptive or hormone replacement therapy comprising a housing, a needle, a pushrod, a needle guiding means, a protective cover and an actuator. The applicator comprises a mechanism wherein needle is crimped such that it help to secure the implant inside the needle. Further, the invention provides a mechanism wherein implant is loaded into the applicator through a slot present in the needle at an angle perpendicular to the longitudinal axis of the needle.

### TECHNICAL FIELD

The present disclosure relates to the field of contraception and hormone replacement therapy. More particularly the disclosure relates to an applicator for inserting an implant, in particularly a rod-like implant containing an active substance(s) under the skin of a human or animal. The applicator comprises a housing, a needle and a penetration guide. The disclosure further relates to a method of loading an implant in the applicator.

### BACKGROUND

There are implantable drug delivery systems approved by Food and Drug Administration (FDA) like IMPLANON® by Organon or NEXPLANON® by Organon for prevention of pregnancy in women with different types of applicators. IMPLANON® and NEXPLANON® are contraceptive implants containing etonogestrel that are inserted in the human body for periods up to 3 years.

Various patents and patent applications disclose different types of applicator systems for inserting an implant into a human or animal body. U.S. Pat. No. 4,223,674 discloses an implant gun including a grip or handle (10), slideably connected to an intermediate member (12), and a hollow needle (16).

EP 596 161 as described in the abstract discloses an apparatus for subcutaneous introduction of a needle (2) into a living being. Guiding means (4,5) are provided at both sides of the needle.

WO 01/68168 as described in the abstract discloses a disposable device for inserting one or several implants, said device comprising a tubular cannula (10) provided with a tip (11), said cannula also serving as a container for the implants, a plunger (20), and a handle (30) having a first end (31) directed towards the cannula (10) and a second end (32) directed away from the cannula.

U.S. Pat. No. 5,695,463 as described in the abstract discloses an injection device for intramuscular or subcutaneous injection of solid or semi-solid medicaments. The device includes a main body member having a needle attached thereto. A protective sleeve covers the needle and retracts into the main body member when the device is pressed against the skin of a patient.

EP 0 304 107 patent as described in the abstract discloses an injection device (denoted by numeral 1), in particular for once-only use, for injecting an implant (6) which can release a drug in a controlled manner, which device comprises a housing (2) which is provided at the injection end with an injection needle (3) in which the implant (6) can be disposed and in which a passage opening is disposed at the actuating end of the housing for a plunger (7,8), mounted in the housing and displaceable in the axial direction of the needle (3), which plunger, on the one hand, can interact with the implant (6) and, on the other hand, is provided with an actuating element, which element is constructed as an element (10) for pressing and supporting against or on the part of the body to be treated.

U.S. Pat. No. 4,820,267 as described in the abstract discloses a device for subcutaneous implantation of single and plural elongated medicament pellets comprising a single dosage where magazine feeding is not applicable because considerations of sterility and cross-contamination require a fresh needle and obturator for each patient. The device includes a cannula supported at a proximal end thereof by a hub which slides within a tubular barrel, the barrel supporting an obturator which selectively penetrates the cannula to maintain an implanted pellet in position as the cannula is withdrawn. For single pellet dosages, the pellet is carried in the fore part of the cannula, while in the case of multiple pellet dosages, the additional pellets, prior to loading, are carried in open-ended cylindrical tubes engageable with a proximal end of the hub whereby the obturator may be employed to transfer the pellet to the cannula from the sleeve which is discarded. Repositioning of the hub within the sleeve is then accomplished without disengagement of the distal end of the cannula from the tissues of the patient and additional implantations may then be performed.

WO 2004/089458 as described in the abstract discloses a device for inserting implantable objects beneath the skin of a patient, including a handle for grasping the device and a base connected to the handle. The base comprises a post, a cannula, and a flexible actuator positioned in an angled track. The cannula is positioned coaxially around and is longitudinally slidable over the post from an extended position, where an implantable object is retained in the cannula, to a retracted position, where the implantable object is released from the cannula.

EP 1 300 173 relates to a hand held implanter for containing and depositing a subcutaneous implant beneath the skin of a patient. FIGS. 11 to 13 illustrate one preferred method for loading the implant (18) into the implanter (110) in the case where the implanter is not preloaded by employing an implant containing vial (90). The vial (90) maintains the implant in a sterile condition during transportation, storage, and loading.

U.S. Pat. No. 9,757,552 as described in the abstract discloses an applicator for inserting an implant, in particular a rod-like implant containing an active substance, under the skin of a human or animal, comprising a housing, a cannula, a cannula holder, an implant accommodated inside the cannula and/or the cannula holder, a protective cover for the cannula, and a mechanism which, at least after the cover has been removed from the cannula (6), secures the implant inside the cannula (6) and/or cannula holder. The mechanism disengages the implant during insertion of the cannula or after the cannula has been inserted. Substantially no lateral force will be exerted during the expelling of the implant from the cannula.

U.S. Pat. No. 8,888,745 as described in the abstract discloses an applicator (1) for inserting an implant, in particular a rod-like implant (2) containing an active substance, under the skin of a human or animal, comprising a housing (3), a cannula (6) extending from the housing (3), and a handle (15) for grasping and maneuvering the applicator (1) and the cannula (6) during insertion of an implant (2). In accordance with the invention, the handle (15) extends above at least part of the length of the cannula (6). Such a handle facilitates insertion of the cannula and/or accurate positioning of the implant.

U.S. Pat. No. 10,092,739 as described in the abstract discloses a kit for assembling a disposable applicator for inserting an implant, in particular a rod-like implant containing an active substance, under the skin of a human or animal, the kit comprising a first component, in turn comprising a main housing part providing a handle for grasping and maneuvering the applicator, a cannula, and a cannula holder mounted in the main housing part, the main housing part having an opening which allows introduction of an implant into the proximal end of the cannula or the cannula holder, and, a second component for closing said opening, in turn comprising a second housing part and a rod attached to or forming an integral whole with the second housing part and mountable inside the cannula or the cannula holder.

Although implantable delivery systems are known in the art, there is still a need to develop improved implantable drug delivery systems. For example, there is a need for an applicator that is more convenient for practitioners and patients from an administration convenience perspective. As another example, there is a need for an applicator which can serve to reduce the risk of damaging the delicate implant while introducing the implant into a needle. This may be the case where the applicator comprises intricate design features to enhance e.g. ergonomics and/or operation safety, and to avoid damage to a delicate implant due to sharp edges of the applicator and/or needle while introduction of the implant into the needle. As a further example, there is a need for an applicator that allows for a more unobstructed view of the implantation site by practitioners during subdermal administration than the currently available subdermal implant applicators.

### SUMMARY

In accordance with one aspect disclosed herein, an applicator for inserting an implant under the skin of a human or animal comprises a housing, a needle, a pushrod, a needle guiding means, a protective cover and an actuator. In accordance with an aspect disclosed herein, the needle guiding means is transparent.

In accordance with another aspect disclosed herein, an applicator for inserting an implant under the skin of a human or animal comprises a housing, a needle, a pushrod, a needle guiding means, a protective cover, an actuator and a mechanism wherein needle is crimped such that one or more needle crimps help to secure the implant inside the needle before insertion thereby preventing the implant accidently falling from the needle when the protective cover is removed.

In accordance with an aspect, an applicator wherein a needle guiding means is transparent. In accordance with another aspect disclosed herein, an applicator for inserting an implant under the skin of a human or animal, comprising a housing, a needle, a pushrod, a needle guiding means, a protective cover, an actuator and a mechanism wherein implant is loaded into the applicator through a slot present in the needle, wherein a needle guiding means is transparent.

In accordance with another aspect disclosed herein, an applicator for inserting an implant under the skin of a human or animal, comprising a housing, a needle, a pushrod, a needle guiding means, a protective cover, an actuator and a mechanism wherein implant is loaded into the applicator through a slot present in the needle at an angle perpendicular to the longitudinal axis of the needle, wherein a needle guiding means is transparent.

In accordance with another aspect disclosed herein, an applicator for inserting an implant under the skin of a human or animal, comprising a housing, a needle, a pushrod, a needle guiding means, a protective cover, an actuator and a mechanism wherein the needle is crimped such that one or more needle crimps help to secure the implant inside the needle before insertion thereby preventing the implant accidently falling from the needle when the protective cover is removed, wherein the implant is loaded into the applicator through a slot present in the needle and wherein a needle guiding means is transparent.

In accordance with another aspect disclosed herein, an applicator for inserting an implant under the skin of a human or animal, comprising a housing, a needle, a pushrod, a needle guiding means, a protective cover, an actuator and a mechanism wherein the needle is crimped such that one or more needle crimps help to secure the implant inside the needle before insertion thereby preventing the implant accidently falling from the needle when the protective cover is removed, wherein a needle guiding means is transparent, wherein the implant is loaded into the applicator through a slot present in the needle at an angle perpendicular to the longitudinal axis of the needle.

In accordance with another aspect disclosed herein, an applicator for inserting an implant under a skin of a human or animal can comprise a housing, a needle extending distally from the housing, the needle being configured to receive the implant, a pushrod within the housing extending within the needle, a needle penetration guide extending distally from the housing along at least a portion of a length of the needle, a protective cover configured to engage the housing to cover the needle and the needle penetration guide, and an actuator provided on the housing configured to be actuated by a user to release the implant from the needle, wherein the needle is crimped such that one or more needle crimps help to retain and secure the implant inside the needle before insertion of the applicator, thereby preventing the implant from accidently falling out of the needle, and wherein the needle comprises a slot provided between a proximal end and a distal end of the needle configured for loading of the implant into the needle through the slot.

The applicator of the preceding paragraph or in other embodiments can include one or more of the following features. The needle penetration guide can be transparent. The slot can be configured for loading of the implant at an angle perpendicular to a longitudinal axis of the needle. A length of the slot can be greater than a length of the implant. The actuator can be configured to be actuated to retract the needle into the housing. The housing can comprise a left side housing, right side housing, and bottom housing. The needle penetration guide can be coupled to a portion of the housing and extends distally from the housing. The needle penetration guide can be configured to extend from the housing substantially parallel to the needle.

In accordance with another aspect disclosed herein, an applicator for inserting an implant under a skin of a human or animal can comprise a housing, a needle configured to receive the implant and deliver the implant under the skin, wherein the needle comprises a first position where the needle extends distally from the housing and a second position where the needle is retracted into the housing, wherein the needle is configured to allow the implant to remain under the skin when the needle moves from the first position to the second position, a needle penetration guide extending distally from the housing along at least a portion of a length of the needle, wherein the needle penetration guide is configured to guide the needle while it is inserted under the skin, an actuator configured to move the needle form the first position to the second position, and a slot in a sidewall of the needle between a proximal end and a distal end of the needle configured for loading of the implant into the needle through the slot.

The applicator of the preceding paragraph or in other embodiments can include one or more of the following features. A length of slot is greater than a length of the implant. The applicator can further comprise a protective cover configured to be releasably engaged and disengaged with the housing, wherein the protective cover is configured to cover the needle and the needle penetration guide when the applicator is not in use. The needle penetration guide can be coupled to a portion of the housing and extends distally from the housing. The needle penetration guide can be configured to extend from the housing substantially parallel to the needle. The needle penetration guide can be transparent.

In accordance with another aspect disclosed herein, an applicator for inserting an implant under a skin of a human or animal can comprise a housing, a needle configured to receive the implant and deliver the implant under the skin, wherein the needle comprises a first position where the needle extends distally from the housing and a second position where the needle is retracted into the housing, wherein the needle is configured to allow the implant to remain under the skin when the needle moves from the first position to the second position, a needle penetration guide coupled to a portion of the housing and extending distally from the housing, wherein the needle penetration guide is configured to guide the needle while it is inserted under the skin, wherein the needle penetration guide can comprise a first portion comprising a solid flat surface on a needle facing side of the penetration guide, wherein the first portion is connected to the housing and extends distally from the housing and substantially parallel to the needle, a second portion positioned distal to the first portion, wherein the second portion comprises a solid curved surface curved away from the needle, wherein the needle penetration guide is transparent; and an actuator configured to move the needle form the first position to the second position.

The applicator of the preceding paragraph or in other embodiments can include one or more of the following features. The applicator can further comprise a protective cover configured to be releasably engaged and disengaged with the housing, wherein the protective cover is configured to cover the needle and the needle penetration guide when the applicator is not in use.

Any feature, structure, or step disclosed herein can be replaced with or combined with any other feature, structure, or step disclosed herein, or omitted. Further, for purposes of summarizing the disclosure, certain aspects, advantages, and features of the disclosure have been described herein. It is to be understood that not necessarily any or all such advantages are achieved in accordance with any particular embodiment disclosed herein. No individual aspects of this disclosure are essential or indispensable.

### BRIEF DESCRIPTION OF THE DRAWINGS

FIG. 1 shows an isometric view of an applicator device.

FIG. 2 shows an isometric view of the applicator device of FIG. 1, with the protective cap removed.

FIG. 3 shows a top view of the applicator device of FIG. 2.

FIG. 4 shows a side view of the applicator device of FIG. 2.

FIG. 5 shows an exploded view of the applicator.

FIG. 6 shows a cross sectional side view of the applicator when the needle is in an extended position.

FIG. 7 shows a sectional side view of the applicator when the needle is in a retracted position.

FIG. 8 shows a locking mechanism when the needle is fully retracted.

FIG. 9 shows a side cross sectional view of a needle.

FIG. 10 shows a perspective bottom view of an applicator receiving an implant.

FIG. 11 is a cross sectional view of an applicator immediately after an implant is placed in the needle.

### DETAILS DESCRIPTION

The present disclosure relates to an applicator for subdermal insertion of a drug delivery device. In some aspects the disclosure relates to an applicator for the administration of actives subdermally for contraception or as hormone replacement therapy. In some cases, the drug delivery device is particularly in the form of an implant. In some cases, the present disclosure relates to X-ray visible implants.

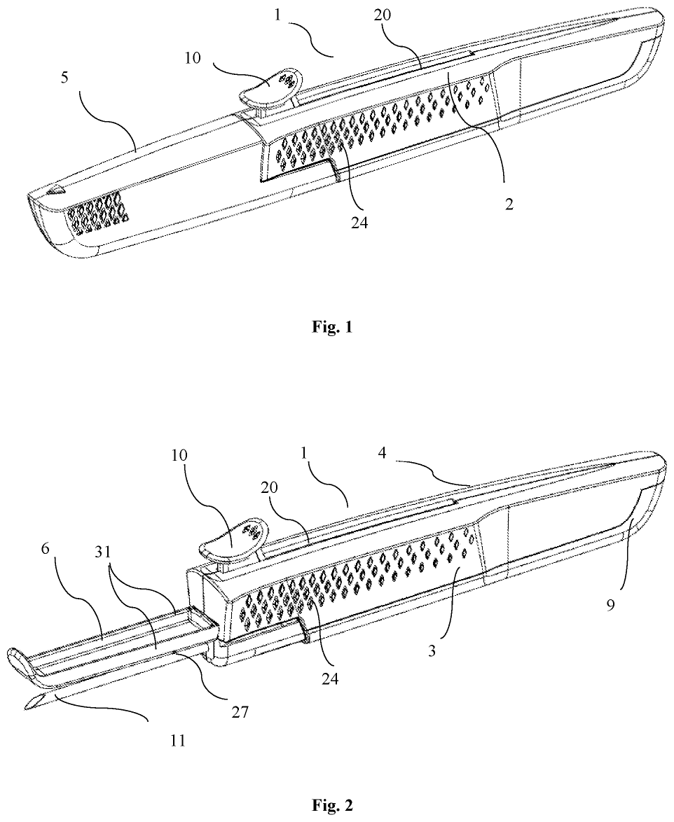

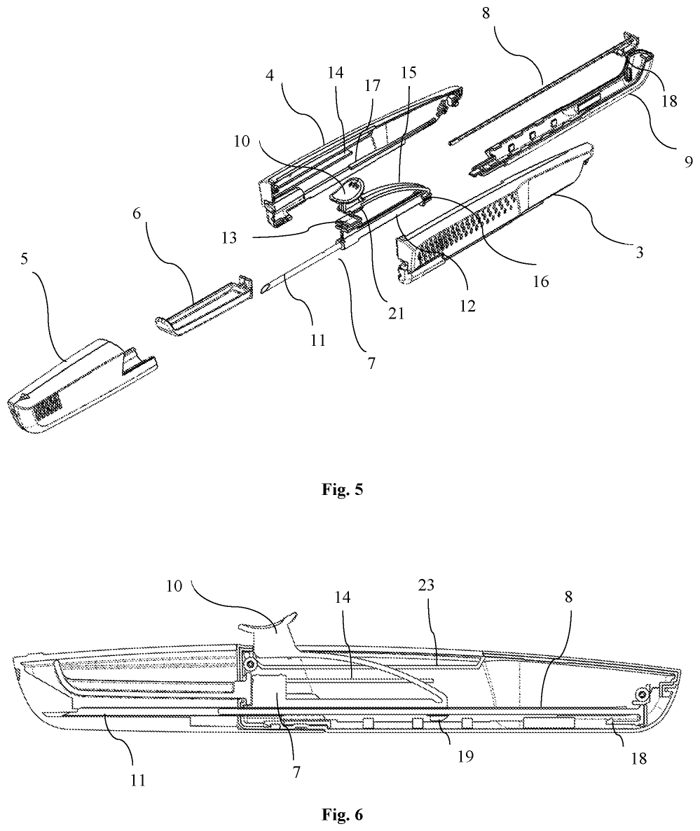

As used herein, the singular form “a”, “an”, and “the” includes plural references unless clearly indicated otherwise and use of other singular forms include the plural and vice versa. An applicator is disclosed herein for inserting an implant, in particular a rod-like implant containing an active substance under the skin of a human or animal. The applicator 1 is illustrated in the perspective view in FIG. 1. The applicator 1 includes a housing 2, a needle 11 (FIG. 2), a pushrod 8 (FIG. 5), a needle guiding means 6 (FIG. 2), a protective cover 5, and an actuator 10. The actuator 10 is present on the top of the housing. The actuator 10 may include ridges, grooves or ribs on the forward surface that are engaged by the user's finger. The housing 2 acts as a handle for grasping the applicator during insertion of an implant. Housing grip 24 allows the user to pull up and advance the needle subdermally, and may include ridges, grooves or ribs on the sides of the housing 2.

As used herein, a needle guiding means can also be called as needle penetration guide and vice-versa.

FIG. 2 is an isometric view of the applicator with the protective cover removed. The protective cover 5 (FIG. 1) is removed to expose the needle 11 and needle penetration guide 6. The protective cover 5 can be releasably engaged and disengaged with the housing. The protective cover 5 is configured to cover the needle 11 and the needle penetration guide 6 when the applicator is not in use.

The penetration guide 6 may be coupled to any portion of the applicator housing 2 in a manner such that it may be coupled reversibly or irreversibly in order to perform the intended function. The penetration guide may be molded from the same material as the housing 2 or may be attached as a separate element or material to the housing 2, e.g. via heat treatment, interlock or snap fit. The penetration guide 6 in some cases may comprise any design that may be maintained substantially parallel to the needle. The penetration guide 6 may have a width measured from one side of the penetration guide to the other side of the penetration guide. The width of the penetration guide 6 can be narrower or wider than the needle. The penetration guide 6 may have a length that extends from the distal tip of the penetration guide to the proximal end of the penetration guide that meets or is attached to the housing 2. The length of the penetration guide 6 can be longer, shorter or of the same length as of the needle.

The penetration guide may comprise a plurality of substantially parallel extensions from the housing 2, each substantially parallel to the needle. In one case, the penetration guide is a single extension as shown in FIGS. 2-4. The penetration guide may also be designed to allow only a single distance between itself and the needle or it may be adjustable to allow the practitioners to set the distance between the two according to the depth of insertion. The gap between penetration guide and the needle may be between about 1 mm to about 10 mm, more preferably between about 1.5 mm to about 5 mm, and most preferably by a distance of between about 1.5 mm to about 3 mm. The gap between the penetrate guide and the needle may be constant along the length of the needle, or it may increase from proximally-to-distally. In some cases, the guide is a solid and may be transparent, translucent or opaque. In some cases, the penetration guide is transparent. A transparent penetration guide allows an unobstructed view of the implantation site during implant administration. In order to control the depth of insertion, the penetration guide has sufficient stiffness to prevent bending of the penetration guide during administration. The penetration guide can deflect not more than 1 mm on application of 1 Newton forces at the end of their straight portion i.e. free end of the penetration guide, preferably the penetration guide can deflect not more than 0.75 mm, more preferably the penetration guide can deflect not more than 0.60 mm on application of 1 Newton forces at the end of their straight portion.

The needle penetration guide 6 is coupled to a portion of the housing and extends distally from the housing as shown in FIG. 4. The needle penetration guide 6 comprises an elongate member. The needle penetration guide can guide the needle while it is inserted under the skin. The needle penetration guide 6 in some cases can include a first portion comprising a flat, plate-like element extending distally from the housing over a majority of the length of the needle. The plate-like element comprises a solid, flat lower surface on a needle facing side of the penetration guide 6, and an opposite upper surface that may be parallel to the lower surface. The plate-like element may have a substantially rectangular transverse cross-section. The plate-like element may be transparent or translucent as described above. The first portion may further comprise ribs 31 extending perpendicularly and/or upwardly from sides of the plate-like element. These ribs 31 may provide further rigidity to the penetration guide 6. The ribs 31 may extend along the left and right edges of the flat, plate-like element of the first portion as shown in FIGS. 2 and 4. In other cases, the rib can be located on a central portion (not on the edges) of the flat (not shown), plate-like element of the first portion. The first portion can be connected to the housing and extend distally from the housing and substantially parallel to the needle. In some cases, a proximal wall extending perpendicularly and/or upwardly from a proximal side of the plate-like element may be attached to the housing.

The first portion of the penetration guide can have a height measured from a first needle facing side of the penetration guide to an opposite second side. The height of the first portion of the penetration guide may remain constant for all or a majority of the length of the first portion or the penetration guide.

The needle penetration guide 6 can include a second portion positioned distal to the first portion. The second portion can include a solid curved surface curved away from the needle. The solid curved surface of the second portion may comprise an extension of the plate-like element of the first portion. The solid curved surface of the second portion may terminate in a rounded distal tip. The second portion of the needle penetration guide 6 may extend to or distally beyond the needle 11.

The shape of the distal end of the penetration guide or second portion of the penetration guide may be configured to prevent extremely steep needle angles relative to the skin from being initiated for initial needle insertion. Further, the planned implant site may be fully visible through the transparent penetration guide, if utilized, throughout needle advancement and administration procedure. The distal end of penetration guide may also provide an indication as to where the initial needle insertion will be initiated as well as providing a perspective as to where the implant will be deployed. For assisting the user in maintaining tissue depth of the implant during the administration procedure, the ventral or lower side of a transparent penetration guide provides a guide for maintaining needle advancement depth in addition to being able to easily observe the administration procedure throughout the needle advancement.

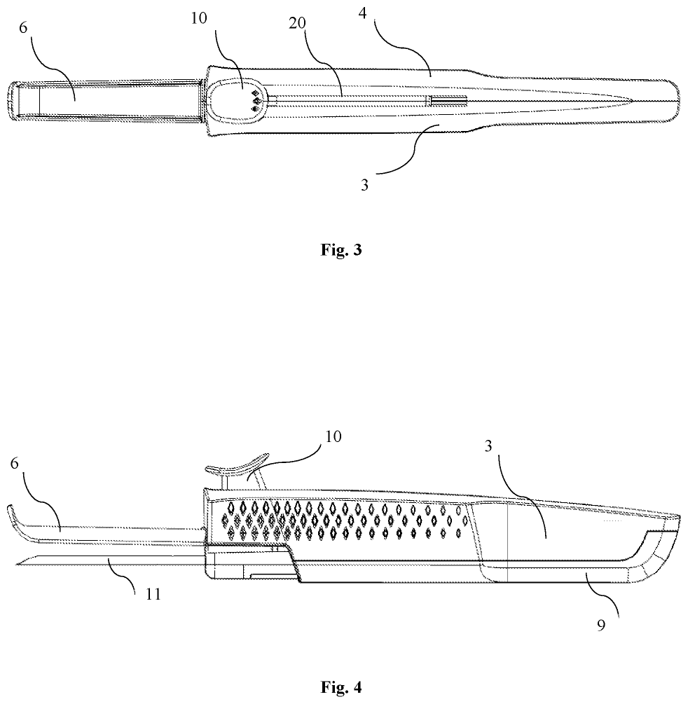

FIG. 3 and FIG. 4 are views of an applicator from top and left side respectively.

FIG. 5 is an exploded isometric view of the applicator. The applicator has a housing 2 which is made up of three different components namely left side housing 3, right side housing 4, and bottom housing 9. Needle subassembly 7 is made of needle 11, needle holder 12, needle guiding means front 13, needle guiding means rear 16, needle assembly pins 19 (FIG. 6), and an actuator 10 attached to the needle holder 12 via flexible strap 15. The needle 11 is fixed to a needle holder 12, which is slidably received inside the housing 2 via housing guides front 14 and the housing guides rear 17. The needle 11 is a hollow rod-like instrument to deliver the implant under the skin of a human or animal. The implant is present in the hollow interior of the needle 11. The needle guiding means front 13 and needle guiding means rear 16 and the housing guides front 14 and the housing guides rear 17 engage respectively to keep needle holder in place during needle insertion while allowing needle holder 12 to slide inside the housing when actuated by actuator 10, i.e. retracted. The housing guides front 14 and housing guides rear 17 are also present on left housing 3 thereby securing needle holder in its place firmly (not shown). The housing guides may be a rib like structure. The pushrod 8 is assembled with the bottom housing 9. The pushrod 8 may be fitted with the bottom housing 9 via molding or snap fit technique. Pushrod 8 may have snap fit finger 18, which is described further in detail below. The pushrod 8 includes a blunt end 29 for engaging the implant. As shown in FIG. 8 the diameter of the pushrod 8 is tapered 30 immediately after blunt surface as in order to facilitate needle locking after needle assembly 7 is retracted.

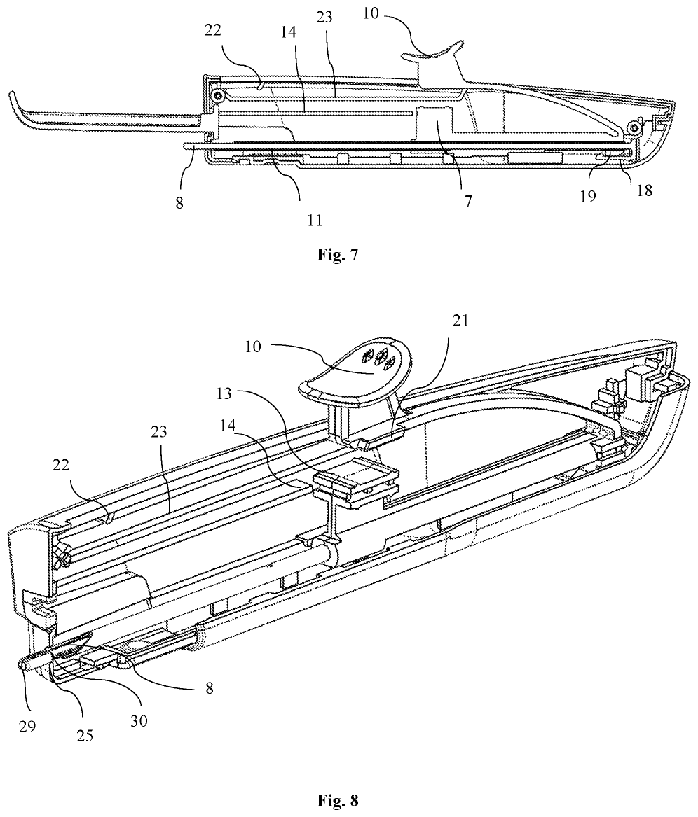

FIG. 6 and FIG. 7 are cross sectional side views of the applicator when the needle is in extended position and retracted positions, respectively. The actuator 10 has flange 21 (FIG. 5 and FIG. 8) which interacts with the detent ribs 22 on the left housing 3 (not shown) and right housing 4 (FIG. 7) to prevent the needle from retracting while the needle is inserted during the administration procedure. The flange 21 and detent ribs 22 have complimentary surfaces to each other. When a user pushes down on the actuator 10, the flange 21 slides along the detent ribs 22 and falls below the detent ribs 22 thereby unlocking the actuator. When the actuator 10 is further pulled back it slides longitudinally along the track 20 (FIG. 1 and FIG. 2) that is formed by left housing 3 and right housing 4 and the needle assembly 7 gets retracted. The housing rib 23 formed in the left housing 3 (not shown) and right housing 4 guide the actuator 10 and restrict further downward movement of the actuator 10 during the retraction procedure. At the end of travel of the actuator 10, needle assembly pins 19 flexes past the back of the pushrod snap finger 18 and locks the needle assembly 7 in place. The arrangement may be made to provide an audible click at the end of travel signaling the user that the implant has been deployed. Further, at the end of travel of the actuator 10, the needle guiding means front 13 disengages from the housing guide front 14. After this disengagement due to the tapered 30 region of a pushrod, the needle assembly 7 is able to move down with respect to the pushrod and when the actuator 10 is pushed forward, it will meet resistance from the front of the needle guiding means front 13 misalignment due to disengagement with housing guide front 14 and the bevel of the needle 11 interfering with the inner wall of the housing just below the needle outlet aperture 25 as shown in FIG. 8. These features provide a means to insure that the needle is fully retracted and made safe within the housing after the administration procedure to prevent accidental needle sticks injury to users.

The needle can receive the implant and deliver the implant under the skin. The needle can have a first extended position where the needle extends distally from the housing and a second retracted position where the needle is retracted into the housing. The needle can allow the implant to remain under the skin when the needle moves from the first extended position to the second retracted position.

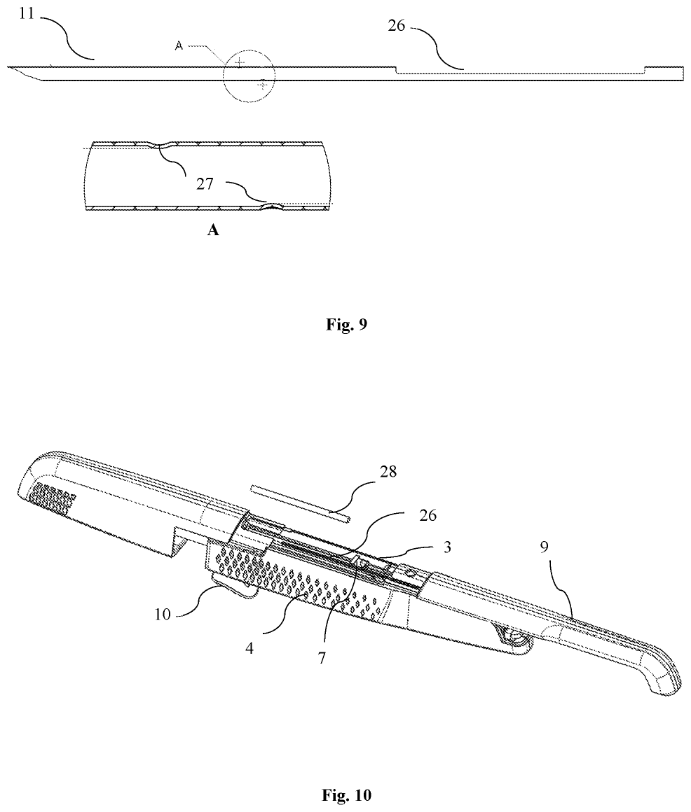

FIG. 9 is a side cross-sectional view of the needle. In some cases, the needle 11 includes one or more inwardly deformed crimps as an implant retaining mechanism. A deformed crimp portion 27 of the needle 11 applies continuous pressure on the implant against the inner wall of the needle retaining the implant in the needle. The deformed crimps 27 may be in the form of a bubble, a dome, a slit, a dimple or the like. The continuous pressure provides a force which is sufficient to secure an implant in the needle while transporting and handling before the insertion. The deformed crimps provide a force or resistance to hold to an implant in order to prevent the implant from falling out of the needle, even if the applicator is rotated 90 degrees so the needle tip is facing downwards, until the needle is retracted while allowing an implant to slide within the needle 11 without causing damage to an implant when the needle 11 is being retracted over the implant by actuation of the actuator 10. During retraction of the needle assembly 7 (FIG. 6 and FIG. 7) the blunt end 29 of the pushrod 8 prevents the implant 28 (FIG. 8) from being retracted; ultimately deploying the implant to its position under the skin. Multiple deformed crimps can be used to provide further assurance that implants will be retained regardless of implant orientation inside the needle. The implant retaining mechanism is located at any position on the needle 11 to serve the desire purpose. In some cases, a needle 11 includes one dimple as an implant retaining mechanism, for example, on the dorsal side of the needle. In some cases, a needle 11 includes two dimples 27 as an implant retaining mechanism. The two dimples 27 are located at different positions on the needle 11, for example, on the dorsal and ventral sides of the needle and, for example, the proximal dimple being on dorsal side. In an example, two dimples 27 can be spaced 180 degrees apart radially. In some examples, two dimples 27 can be spaced 180 degrees apart radially and 2 mm to 10 mm apart axially, preferably 4 mm apart axially as shown in exploded view A of FIG. 9. In another case, a needle 11 includes three dimples as an implant retaining mechanism. In some cases, three dimples coplanarly spaced 120 degrees apart on the needle circumference. A depth of deformed crimp portion 27 is in the range from about 0.01 mm to about 0.2 mm, more preferably from about 0.025 mm to about 0.15 mm, more preferably about 0.025 to about 0.1 mm. Each deformed crimp portion 27 may have different depth irrespective of the number of deformed crimp portion 27 on the needle 11.

In order to facilitate the implant insertion in the needle 11, a large slot 26 is provided in the needle. The slot 26 is either molded or cutout. In some cases, a slot 26 is a 180 degree cutout with a length which is more than the length of the implant. In some cases, a slot is of about 10 mm to about 60 mm in length, more preferably a slot is of about 15 mm to about 45 mm in length. In some cases, the slot is provided in between two ends of the needle (between a proximal end and a distal end of the needle). In some cases, the slot is present at the proximal portion of the needle which is opposite to the sharp end or bevel i.e. distal end, and on the ventral side of the needle as can be seen in FIG. 9. For example, the slot is positioned in a sidewall of the needle between a proximal end and a distal end of the needle and the implant can be loaded into the applicator through the slot provided in the sidewall of the needle between two ends of the needle.

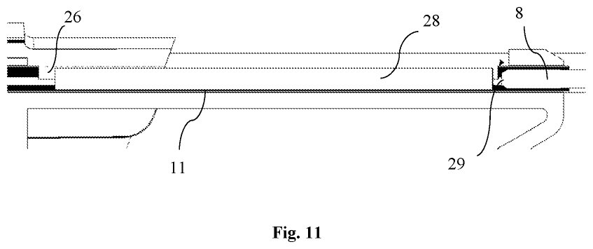

FIG. 10 shows a perspective view of an applicator receiving an implant. Before insertion of the implant the applicator is either a kit or preassembled applicator. When in the form of a kit, a bottom housing 9 remains a separate component and is not assembled to the applicator leaving access to the slot 26 on the needle assembly 7. When the applicator is preassembled it is present as a single piece. The bottom housing 9 is separated from the preassembled applicator to give access to the slot 26 on the needle assembly 7 just before the insertion of the implant 28. The applicator assembly is then oriented upside-down and the implant 28 is dropped into the slot 26 of the needle 11. FIG. 11 is a cross section view of an applicator showing an implant immediately after it is placed in the needle 11 through the large slot 26. Bottom housing 9 is then slid into side housings 3 and 4 with pushrod 8 incorporated into the bottom housing 9 pushing the implant 28 into correct, pre-deployment position and the housing is fully closed. When fully assembled the pushrod 8 is partially located inside the needle 11. The loading of an implant can be advantageous as an implant is directly introduced into the needle to prevent the damage of a delicate implant which otherwise may cause damage if it is placed directly in the end of the applicator housing or needle holder assembly and then pushed into the needle. The implant 28 is directly dropped lengthwise at an angle perpendicular to the longitudinal axis of the needle 11 into the slot 26 of the needle 11 thereby avoiding the consequential damage.

In an example, an applicator is especially suitable for use with implants that slowly release an active substance over an extended period of time. An implant of this type, which can release a contraceptive agent or hormone replacement agent in virtually constant quantities over a period of at least 2 years, and, in some cases, for about 3 to 5 years. An example of such an implant is a single-rod contraceptive implant that provides protection against pregnancy for an extended period of time, for example 3 years. The implant is cylindrical or virtually cylindrical with a diameter about 1 mm to about 3 mm, and, in some cases, about 2 mm, and possesses a variable length. The length may be between 10 mm and 50 mm. The contraceptive active substance, which can be employed in the implant is a therapeutically effective amount of highly active progestagen, particularly 3-keto-desogestrel medroxyprogesterone, levonorgestrel or gestodene. 3-keto-desogestrel also known as etonogestrel. These contraceptive substances are highly active substances which show already an effective progestational action with a daily dosage of about 15-100 μg. In one case, an implant comprises of a non-biodegradable rod measuring nominally 40 mm in length and 2 mm in diameter and 3-keto-desogestrel. After insertion, the rod slowly releases 3-keto-desogestrel for a period of 2 to 5 years. The implant may contain a radio-opaque element such as barium sulphate, titanium oxide, bismuth oxide, tantalum, tungsten, or platinum. The radio-opaque agent facilitates viewing of the implant during insertion and at any point while the device is implanted.

The hormone replacement agents that can be employed in the implant include an estrogen and progesterone components. Estrogen therapies are numerous, and include those indigenous to the human ovary, for example, estradiol, estriol, conjugated equine estrogen (CEE). Progesterone component includes either a synthetic version of the hormone progesterone (such as dydrogesterone, medroxyprogesterone, norethisterone and levonorgestrel), or a version called micronised progesterone (sometimes called body identical, or natural) that is chemically identical to the human hormone.

When in use, a medical professional can take the applicator 1 in one hand, e.g. with the thumb on one side of the housing and the fingers on the other side, and insert the needle 11 under the skin of a patient. The applicator 1 may be held on the left and right side housing 3 and 4 portions of the housing proximal to the needle penetration guide 6. During insertion, transparent penetration guide 6 enables clear view for the medical professional of the needle 11, the planned implant site, and advancement and administration procedure. Subsequently, after insertion, the actuator 10 is unlocked and is pulled rearwards, for example with the index finger of the hand that holds the applicator, and the needle 11 is retracted from the skin of the patient.

The figures illustrates aspects of the present disclosure, and is set forth to assist in understanding the present disclosure. This example should not be construed as specifically limiting the present disclosure described and claimed herein. Variations of the present disclosures, including the substitution of all equivalents now known or later developed, which would be within the purview of those skilled in the art, and changes in formulation or minor changes in experimental design, are considered to fall within the scope of the present disclosure and appended claims.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

The embodiments and examples disclosed herein are illustrative of the principles of the disclosure. Other modifications that may be employed are within the scope of the present disclosure. Thus, by way of example, but not of limitation, alternative configurations may be utilized in accordance with the teachings herein. Accordingly, the present disclosure is not limited to that precisely as shown and described.

1. An applicator for inserting an implant under a skin of a human or animal, comprising:
a housing;
a needle extending distally from the housing, the needle being configured to receive the implant;
a pushrod within the housing extending within the needle;
a needle penetration guide extending distally from the housing along at least a portion of a length of the needle;
a protective cover configured to engage the housing to cover the needle and the needle penetration guide; and
an actuator provided on the housing configured to be actuated by a user to release the implant from the needle;
wherein the needle is crimped such that one or more needle crimps help to retain and secure the implant inside the needle before insertion of the applicator, thereby preventing the implant from accidently falling out of the needle; and
wherein the needle comprises a slot provided between a proximal end and a distal end of the needle configured for loading of the implant into the needle through the slot.

2. The applicator according to claim 1, wherein the needle penetration guide is transparent.

3. The applicator according to claim 1, wherein the slot is configured for loading of the implant at an angle perpendicular to a longitudinal axis of the needle.

4. The applicator according to claim 1, wherein the length of slot is greater than the length of implant.

5. The applicator according to claim 1, wherein the actuator is configured to be actuated to retract the needle into the housing.

6. The applicator according to claim 1, wherein the housing comprises a left side housing, right side housing, and bottom housing.

7. The applicator according to claim 1, wherein the needle penetration guide is coupled to a portion of the housing and extends distally from the housing.

8. The applicator according to claim 1, wherein the needle penetration guide is configured to extend from the housing substantially parallel to the needle.

9. An applicator for inserting an implant under a skin of a human or animal, comprising:
a housing;
a needle configured to receive the implant and deliver the implant under the skin, wherein the needle comprises a first position where the needle extends distally from the housing and a second position where the needle is retracted into the housing, wherein the needle is configured to allow the implant to remain under the skin when the needle moves from the first position to the second position;
a needle penetration guide extending distally from the housing along at least a portion of a length of the needle, wherein the needle penetration guide is configured to guide the needle while it is inserted under the skin;
an actuator configured to move the needle from the first position to the second position, and a slot in a sidewall of the needle between a proximal end and a distal end of the needle configured for loading of the implant into the needle through the slot.

10. The applicator according to claim 9, wherein the length of slot is greater than the length of implant.

11. The applicator according to claim 9, further comprises a protective cover configured to be releasably engaged and disengaged with the housing, wherein the protective cover is configured to cover the needle and the needle penetration guide when the applicator is not in use.

12. The applicator according to claim 9, wherein the needle penetration guide is coupled to a portion of the housing and extends distally from the housing.

13. The applicator according to claim 9, wherein the needle penetration guide is configured to extend from the housing substantially parallel to the needle.

14. The applicator according to claim 9, wherein the needle penetration guide is transparent.

15. An applicator for inserting an implant under a skin of a human or animal, comprising:
a housing;
a needle configured to receive the implant and deliver the implant under the skin, wherein the needle comprises a first position where the needle extends distally from the housing and a second position where the needle is retracted into the housing, wherein the needle is configured to allow the implant to remain under the skin when the needle moves from the first position to the second position;
a needle penetration guide coupled to a portion of the housing and extending distally from the housing, wherein the needle penetration guide is configured to guide the needle while it is inserted under the skin, wherein the needle penetration guide comprises:
a first portion comprising a solid flat surface on a needle facing side of the penetration guide, wherein the first portion is connected to the housing and extends distally from the housing and substantially parallel to the needle,
a second portion positioned distal to the first portion, wherein the second portion comprises a solid curved surface curved away from the needle,
wherein the needle penetration guide is transparent; and
an actuator configured to move the needle form the first position to the second position.

16. The applicator according to claim 15, further comprises a protective cover configured to be releasably engaged and disengaged with the housing, wherein the protective cover is configured to cover the needle and the needle penetration guide when the applicator is not in use.

17. The applicator according to claim 1, wherein the first portion of a needle penetration guide further comprise a ribs extending perpendicularly and/or upwardly from the sides of the solid flat surface.

18. The applicator according to claim 1, wherein the penetration guide deflect not more than 1 mm on application of 1 Newton forces at the end of their straight portion.

19. The applicator according to claim 1, further comprises a housing guide front, housing guide rear, a needle guiding means front, a needle guiding means rear and the needle holder; wherein the needle guiding means front and the needle guiding means rear and the housing guides front and the housing guides rear engage respectively to keep needle holder in place during the needle insertion while allowing needle holder to slide inside the housing when actuated by actuator; wherein upon release of the implant from the needle the needle guiding means front disengage with housing guide front to secure the needle within the housing.
